(19) United States
(12) Patent Application Publication
(10) Pub. No.: US 2002/0019652 A1
(43) Pub. Date: Feb. 14, 2002

(54) Two part tens bandage

(75) Inventors: Luiz B. Da Silva, Danville, CA (US); David I. Blatt, Margate, FL (US); Jeffrey S. Mannheimer, Newton, PA (US)

Correspondence Address: John P. Wooldridge, 1334 Ridgestone Court, Livermore, CA, 94551, US

(73) Assignee: Cyclotec Advanced Medical Technologies

(21) Appl. No.: 09/854,297

(22) Filed: May 10, 2001

Related U.S. Application Data
(63) Continuation-in-part of application No. 09/350,426, filed on Jul. 8, 1999.

(51) Int. Cl. A61N 1/36

(52) U.S. Cl. 607/72

(57) ABSTRACT

A sterile bandage is combined with a TENS device for use in covering a wound and providing electrical stimulation to promote healing and block pain.

This is a continuation-in-part of U.S. patent application Ser. No. 09/350,426, titled “Miniature Wireless Transcutaneous Neuro Or Muscular-Stimulation Unit” filed Jul. 8, 1999, and incorporated herein by reference.

### BACKGROUND OF THE INVENTION

1. Field of the Invention

The present invention relates to an integrated bandage and electrical stimulation TENS system, and more particularly, it relates to a treatment electrode device that would provide electric stimulation to an anatomical site that is covered by a bandage.

2. Description of Related Art

For years, bandages have been used to control bleeding and provide a protected sterile environment to promote healing. All cuts and abrasions should be thoroughly cleaned with soap and water to remove foreign material that can lead to infection. Once cleaned the wound should be kept clean and dry. The area should be covered with a bandage (sterile gauze and/or adhesive bandage) to help prevent infection. While a bandage provides necessary wound protection, it does not control pain.

Today, Transcutaneous Electrical Nerve Stimulator (TENS) devices are being used to relieve pain in a variety of ways. Electrodes are placed on the skin above the painful area and mild electrical currents are applied. The stimulation of additional peripheral nerve endings has an inhibitory effect on the nerve fibers generating the pain. TENS is a safe non-invasive drug-free method of pain management.

A wide variety of TENS devices have developed and are generally described in U.S. Pat. No. 6,023,642 by Shealy et al., U.S. Pat. No. 5,620,470 by Gliner et al., U.S. Pat. No. 5,607,454 by Cameron et al., U.S. Pat. No. 5,601,612 by Gliner et al., all of which are incorporated herein by reference. However, none of these references describe an integrated TENS and electrode system.

The parent application, published as WO00103768A1 by Michelson et al. describes a miniature wireless transcutaneous neuro stimulation device that integrates the stimulating unit with the electrodes in a convenient and easy to apply module. However, the device described in WO00103768A1 is not well suited for treating areas with open wounds or cuts.

Therefore, there is a need for an improved device and method for applying electrical stimulation to an anatomical site with an open wound or cut In addition, given the high cost of the electronic stimulation module, it is important to develop a two-component bandage that allows the electronic module to be used with multiple low cost disposable sterile sections. The present invention provides a new and improved device for integrating a treatment electrode into a low cost bandage that can block pain, reduce healing time, and possibly enhance drug delivery.

### SUMMARY OF THE INVENTION

An object of the present invention is to provide a device and method for applying a treatment electrode to an anatomical site covered by a bandage.

The present invention is a device that allows electrical stimulation to an anatomical site that is covered by a bandage. The electrode is applied to block pain and improve healing of a wound. A preferred embodiment of the present invention is comprised of a two part TENS bandage. The bottom portion being a one time use sterile bandage that would connect to the top portion containing the electronics module which could be reused multiple times.

These and other objects will be apparent to those skilled in the art based on the teachings herein. Other objects and advantages of the present invention will become apparent from the following description and accompanying drawings.

### BRIEF DESCRIPTION OF THE DRAWINGS

The accompanying drawings, which are incorporated into and form part of this disclosure, illustrate embodiments of the invention and together with the description, serve to explain the principles of the invention.

FIG. 1 shows how the present invention would be applied to cover a wound.

FIG. 2 shows views of both the top and bottom portions of the invention.

FIG. 3 shows a cross sectional view of the upper part of the device.

FIG. 4 shows a cross sectional view of the lower part of the device.

### DETAILED DESCRIPTION OF THE INVENTION

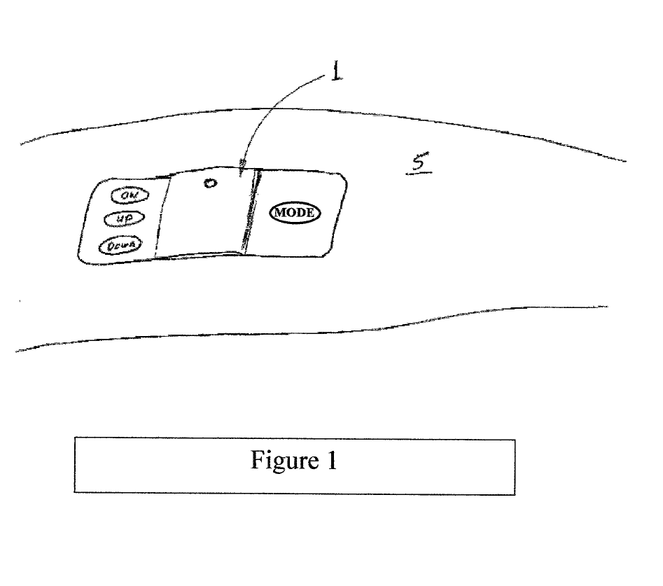

An embodiment of the present invention integrates a treatment electrode with a bandage. FIG. 1 shows how the present invention 1 would be placed on skin 5 to cover a cut or abrasion. The electrical stimulation provided by device 1 would block pain, improve healing, and possibly deliver medication.

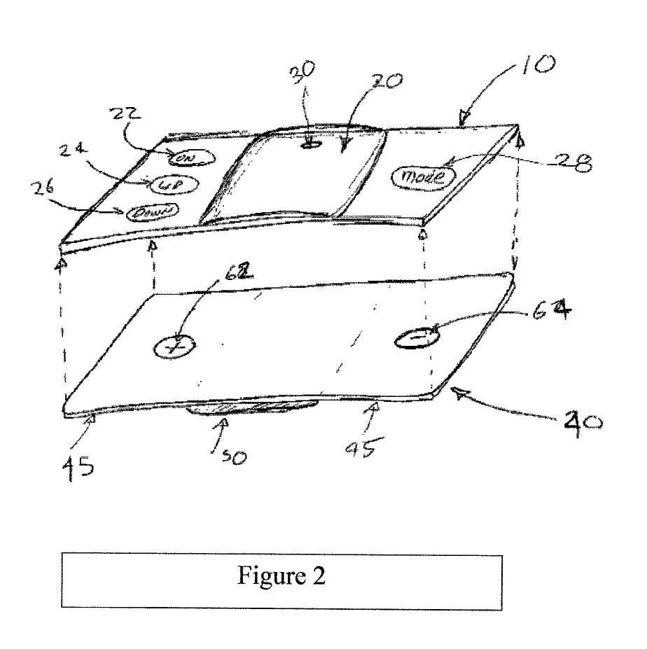

FIG. 2 shows one embodiment of the present invention. The two part TENS bandage is comprised of two components, an upper part 10, and a lower part 40 that attaches to the upper component for normal use. The upper part 10 contains the electronics module 20 and control keys 22, 24, and 26. The user pushes the control key 22 to power the device on and off. The up key 24 and the down key 26 are used to adjust the intensity of the electrical stimulation. The mode key 28 is used to select the stimulation mode.

The lower portion has three main sections. The outer electrode sections 45 have an adhesive lower surface that sticks to the skin. The center section 50 is an absorbent sterile gauze that can contain medication. The top surface of the lower part has electrical conductive pads 62 and 64 that connect the electronics module 20 to the electrode.

In normal use, the lower portion 40 is joined to the upper part 10 of the device and then applied to the treatment area. The user then turns on the stimulation by pressing key 22 and then selects the mode by pressing key 28. In a simpler embodiment, the device would have no mode key; the mode would be preset. The user presses the up key 24 and the down key 26 to adjust the stimulation to a comfortable and effective level. When it is time to clean the wound or replace the lower portion bandage. The user peels the device off the skin and disposes of the lower portion bandage 40 and replaces it with a new sterile bandage. The TENS unit can then be reapplied. An advantage of this design is that the expensive electronics can be reused many times.

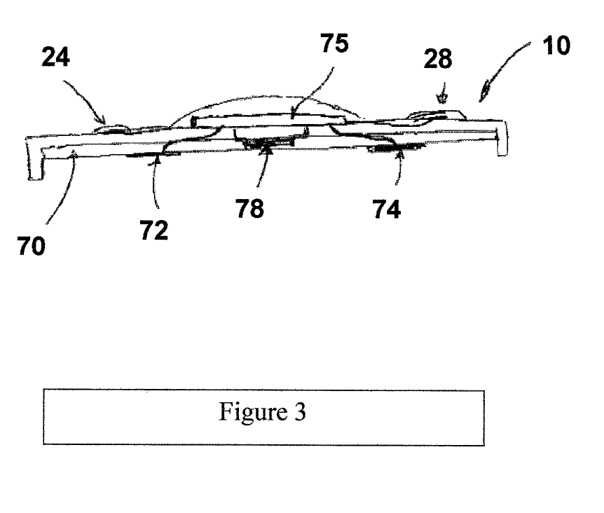

FIG. 3 shows a cross-sectional view through the upper part 10 of the device. The electrical circuit board 75 connects to conductive pads 72 and 74 that connect to pads 62 and 64 when combined with the lower part 40. The inner layer 70 is a thin magnetic polymer that holds the lower part 40 (FIG. 2)in place during normal use. The advantage of this material is that it can be used multiple times with multiple lower bandage parts. A limited use device could use an adhesive layer rather than a magnetic layer and lower portion 40. A replaceable battery 78 powers the electrical circuit board 75. The electrical circuit board 75 can provide a variety of stimulation modes. A suitable electronic module is described in the parent application.

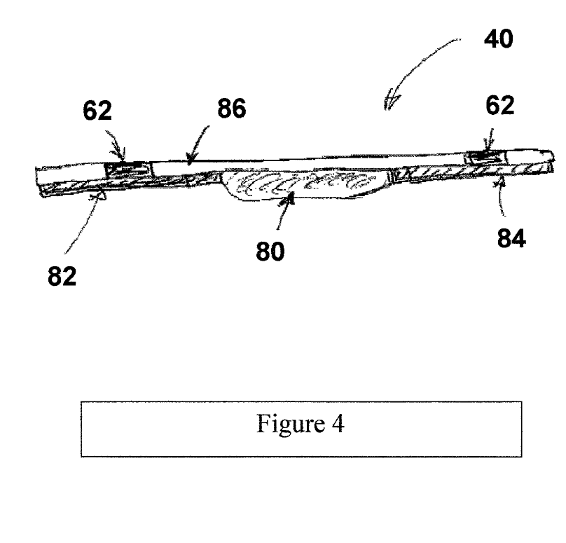

FIG. 4 shows a cross sectional view through the lower part 40. This part of the device comes in contact with the area to be treated and will only be used for one application. The sterile gauze area 80 is absorbent and of the same material used in conventional bandages, medical gauze or surgical dressings. The outer areas 82 and 84 form the electrodes and are made of a conductive adhesive that holds the device in place when applied to skin. In addition, the conductive adhesive when applied reduces skin resistance and allows current to flow between the electrodes. The top layer 86 is made up of a magnetic polymer so that it easily attaches to the upper part 10. The conductive pads 22 and 24 connect to the conductive electrodes 82 and 84.

In an alternative embodiment the upper and lower parts of the device are joined with Velcro. In this case, layer 86 comprises one component layer of Velcro. The bottom layer 70 of the upper part 10 is replaced by the other Velcro component.

A patent application titled “Integrated Cast And Muscle Stimulation System” filed on the same day as this application is incorporated herein by reference.

The above descriptions and illustrations are only by way of example and are not to be taken as limiting the invention in any manner. One skilled in the art can substitute known equivalents for the structures and means described. The full scope and definition of the invention, therefore, is set forth in the following claims.

We claim:

1. An apparatus, comprising:
a bandage; and
a treatment electrode operatively attached to said bandage.

2. The apparatus of claim 1, wherein said treatment electrode comprises Transcutaneous Electrical Nerve Stimulator (TENS).

3. The apparatus of claim 2, wherein said TENS comprises an electronics module and control keys, wherein a user can power the device on and off and adjust the intensity of electrical stimulation provided by said TENS.

4. The apparatus of claim 3, wherein said bandage comprises outer electrode sections that have an adhesive lower surface that sticks to the skin, said bandage further comprising a center section that is an absorbent sterile gauze that can contain medication.

5. The apparatus of claim 4, wherein said bandage has a top portion that has electrical conductive pads that connect said electronics module to said electrode sections.

6. The apparatus of claim 1, wherein said bandage is disposable.

7. A TENS bandage comprising:
a reusable upper part with TENS electronics and user interface;
a disposable lower part with electrodes and sterile gauze; and
a means for joining said upper part to said lower part to achieve contact between said upper part and said lower part.

8. The TENS bandage of claim 1, wherein said means for joining are selected from the group consisting of a magnetic polymer, Velcro and multiuse adhesive layer.

9. A method of treatment of a wound, comprising:
providing a bandage having an integrated treatment electrode operatively attached to said bandage;
applying said bandage to a wound; and
electrically stimulating said wound with electrical energy from said treatment electrode.

10. The method of claim 9, wherein said treatment electrode comprises Transcutaneous Electrical Nerve Stimulator (TENS).

11. The method of claim 10,wherein said TENS comprises an electronics module and control keys, wherein a user can power the device on and off and adjust the intensity of electrical stimulation provided by said TENS.

12. The method of claim 11, wherein said bandage comprises outer electrode sections that have an adhesive lower surface that sticks to the skin, said bandage further comprising a center section that is an absorbent sterile gauze that can contain medication.

13. The method of claim 12, wherein said bandage has a top portion that has electrical conductive pads that connect said electronics module to said electrode sections.

14. The method of claim 9, wherein said bandage is disposable.
